(19) United States
(12) Patent Application Publication
(10) Pub. No.: US 2017/0321666 A1
(43) Pub. Date: Nov. 9, 2017

(54) VARIABLE-STIFFNESS ACTUATOR

(71) Applicant: OLYMPUS CORPORATION, Tokyo (JP)

(72) Inventor: Tetsuya MORISHIMA, Hachioji-shi (JP)

(73) Assignee: OLYMPUS CORPORATION, Tokyo (JP)

(21) Appl. No.: 15/658,684

(22) Filed: Jul. 25, 2017

Related U.S. Application Data
(63) Continuation of application No. PCT/JP2015/052556, filed on Jan. 29, 2015.

(51) Int. Cl. F03G 7/06; A61M 25/00; A61B 1/005

(52) U.S. Cl. CPC F03G 7/065; A61M 25/0043; A61B 1/0055; A61M 2025/0064; A61M 2205/0266; A61M 2025/0058

(57) ABSTRACT

A variable-stiffness actuator capable of providing different stiffnesses for a flexible member includes a shape-memory member that can transit in phase between a first phase and a second phase and an inducing member that causes phase transition between the first phase and the second phase into the shape-memory member. The shape-memory member is arranged in the flexible member with at least one free end. The shape-memory member takes a flexible state in which it is easily deformable by an external force when it is in the first stare, so as to provide lower stiffness for the flexible member. The shape-memory member takes a rigid state in which it tends to take a memorized shape memorized beforehand against an external force when it is in the second stare, so as to provide higher stiffness for the flexible member.

### CROSS-REFERENCE TO RELATED APPLICATIONS

This application is a Continuation Application of PCT Application No. PCT/JP2015/052556, filed Jan. 29, 2015, the entire contents of which are incorporated herein by reference.

### BACKGROUND OF THE INVENTION

### 1. Field of the Invention

The present invention relates to a variable-stiffness actuator for varying the stiffness of a flexible member.

### 2. Description of the Related Art

Japanese Patent No. 3122673 discloses an endoscope in which the stiffness of a flexible portion of an insertion section is allowed to be varied. In this endoscope, a flexible member (e.g. a coil pipe) has both ends fixed at predetermined positions in the endoscope, and a flexibility adjustment member (e.g. flexibility adjustment wire inserted through a coil pipe) is fixed to the flexible member through a separator. The flexible member and the flexibility adjustment member extend to an operation section along the flexible portion and extend almost all over the flexible portion. The flexible member is compressed and stiffened by pulling the flexibility adjustment member, thereby; the stiffness of the flexible portion is varied.

Since the flexible member and the flexibility adjustment member extend almost all over the flexible portion, a very great force is required to drive such a mechanism. To motorize the mechanism, a large-sized motive power source is required and its structure becomes large in scale.

Japanese Patent No. 3142928 discloses a variable-stiffness apparatus for flexible tubes using a shape-memory alloy. The variable-stiffness apparatus includes a coil provided in a flexible tube, an electrical insulative tube provided inside the coil, a shape-memory alloyed wire located in the electrical insulative tube to extend in its axial direction, and an energization heating means to energize the shape-memory alloyed wire.

The shape-memory alloyed wire has the properties of elongating at a low temperature and contracting at a high temperature. The shape-memory alloyed wire extends out through fixed portions at both ends of the coil, and caulking members are fixed to the both ends. The shape-memory alloyed wire is arranged so that it loosens at a low temperature and it tightens up with the caulking members being engaged with the fixed portions at a high temperature.

The shape-memory alloyed wire contracts to stiffen the coil at a high temperature at which it is energized by the energization heating means. On the other hand, the shape-memory alloyed wire elongates to soften the coil at a low temperature at which it is not energized.

Since the variable-stiffness apparatus is simple in structure, it can be miniaturized. However, when the shape-memory alloyed wire contracts, it is restricted at both ends, and a load is applied to the shape-memory alloyed wire. Therefore, the shape-memory alloyed wire has difficulty with its durability.

### BRIEF SUMMARY OF THE INVENTION

A variable-stiffness actuator includes a shape-memory member that can transit in phase between a first phase and a second phase and an inducing member that causes phase transition between the first phase and the second phase into the shape-memory member. The shape-memory member is arranged in the flexible member with at least one free end. The shape-memory member takes a flexible state in which it is easily deformable by an external force when it is in the first stare, so as to provide lower stiffness for the flexible member. The shape-memory member takes a rigid state in which it tends to take a memorized shape memorized beforehand against an external force when it is in the second stare, so as to provide higher stiffness for the flexible member.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 shows a variable-stiffness actuator according to an embodiment.

FIG. 2 shows a variable-stiffness actuator according to another embodiment.

FIG. 3 is an illustration for explaining an operation of a variable-stiffness actuator, showing how the stiffness state of a shape-memory member is varied by switching a switch of a drive circuit.

FIG. 4 is an illustration for explaining an operation of a variable-stiffness actuator, showing how the stiffness state of a shape-memory member is varied by switching a switch of a drive circuit in a situation where an external force is exerted on the vicinity of a free end of the shape-memory member in a direction perpendicular to the central axis of the shape-memory member.

FIG. 5 is an illustration for explaining an operation of a variable-stiffness actuator, showing how the stiffness state of a shape-memory member is varied by switching a switch of a drive circuit in a situation where an external force is exerted on a free end of the shape-memory member in a direction parallel to the central axis of the shape-memory member.

FIG. 6 is an illustration for explaining an operation of a variable-stiffness actuator, showing how the presence and absence of an external force are switched in a situation where a switch of a drive circuit is in an off state and a shape-memory member is in a flexible state.

FIG. 7 is an illustration for explaining an operation of a variable-stiffness actuator, showing how the stiffness state of a bent shape-memory member is varied from a flexible state to a rigid state by switching a switch of a drive circuit.

FIG. 8 is an illustration for explaining an operation of a variable-stiffness actuator, showing how the presence and absence of an external force are switched in a situation where a switch of a drive circuit is in an on state and a shape-memory member is in a rigid state.

### DETAILED DESCRIPTION OF THE INVENTION

### CONSTITUTION EXAMPLE

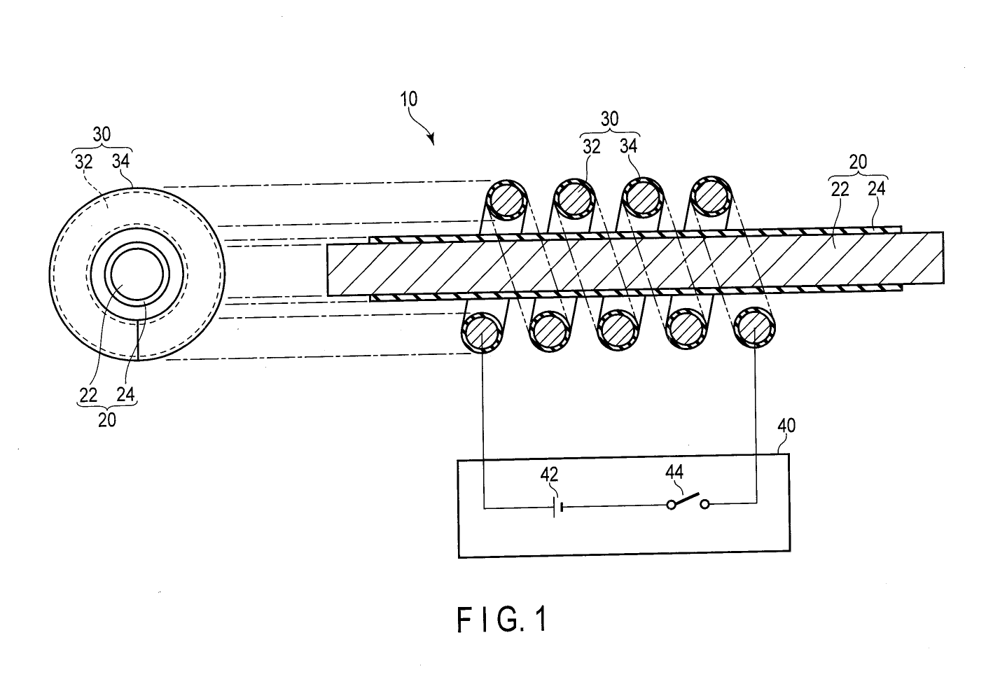

FIG. 1 shows a variable-stiffness actuator according to an embodiment. As shown in FIG. 1, a variable-stiffness actuator 10, which has a function of providing different stiffnesses for a flexible member by taking different stiffness states, includes a shape-memory member 20 that can transit in phase between a first phase and a second phase and an inducing member 30 that causes phase transition between the first phase and the second phase into the shape-memory member 20. The shape-memory member 20 is arranged in the flexible member with at least one free end.

The shape-memory member 20 takes a flexible state in which it is easily deformable by an external force, or it exhibits a low elastic modulus, when it is in the first stare, so as to provide lower stiffness for the flexible member. The shape-memory member 20 takes a rigid state in which it tends to take a memorized shape memorized beforehand against an external force, or it exhibits a high elastic modulus, when it is in the second stare, so as to provide higher stiffness for the flexible member. The memorized shape may be, but not limited to, a linear shape.

Herein, the external force means force that can cause the shape-memory member 20 to be deformed, and gravity is considered to be part of the external force. The inducing member 30 has performance of generating heat.

The shape-memory member 20 has properties of transiting in phase from the first phase to the second phase in response to the heating of the inducing member 30.

The shape-memory member 20 may be constituted chiefly from, e.g. a shape-memory alloy. The shape-memory alloy may be alloy including, but not limited to, e.g. NiTi. The shape-memory member 20 may also be constituted chiefly from another material, but not limited to, such as shape-memory polymer, shape-memory gel and shape-memory ceramics.

Herein, a member being constituted chiefly from a material means that the member as a whole is made of the material and, in addition to this, the member includes not only a member made of the material but also a member made of another material.

The shape-memory alloy that constitutes chiefly the shape-memory member 20 may be, for example, something that transits in phase between a martensitic phase and an austenitic phase. In the martensitic phase, the shape-memory alloy is plastically deformed relatively easily by an external force. In other words, the shape-memory alloy exhibits a low elastic modulus in the martensitic phase. In the austenitic phase, the shape-memory alloy is not easily deformed by an external force. Even though the shape-memory alloy is deformed by a greater external force, it exhibits superelasticiy and returns to its memorized shape when the greater external force is lost. In other words, the shape-memory alloy exhibits a high elastic modulus in the austenitic phase.

The inducing member 30 may be constituted by, e.g. a heater.

In other words, the inducing member 30 may have properties of generating heat upon receipt of current flowing therethrough. The inducing member 30 has only to have performance of generating heat and may be constituted by, but not limited to the heater, an image pickup element, a light guide, another element or member, etc. The inducing member 30 may also be constituted by a structure that generates heat by a chemical reaction.

The shape-memory member 20 may be constituted chiefly from a conductive material. For example, the shape-memory member 20 includes a main body 22 made from a conductive material such as a shape-memory alloy and an insulation film 24 provided around the main body 22. The insulation film 24 serves to prevent a short circuit from occurring between the shape-memory member 20 and the inducing member 30. The insulation film 24 is provided to cover a portion facing at least the inducing member 30. In FIG. 1, the outer surface of the main body 22 is partly covered. Without limiting to this, the outer surface of the main body 22 may be all covered or the main body 22 maybe entirely covered.

The inducing member 30 may be constituted chiefly from a conductive material. For example, the inducing member 30 includes a main body 32 of a conductive material and an insulation film 34 provided around the main body 32. The insulation film 34 serves to prevent a short circuit from occurring between the shape-memory member 20 and the inducing member 30 and a short circuit from occurring between portions adjacent to the main body 32 of the inducing member 30.

The variable-stiffness actuator 10 includes an insulation member to prevent a short circuit from occurring between the shape-memory member 20 and the inducing member 30. The insulation film 24 of the shape-memory member 20 and the insulation film 34 of the inducing member 30 correspond to the insulation member. If the insulation film 34 of the inducing member 30 has a reliable short-circuit prevention function, the insulation film 24 of the shape-memory member 20 maybe omitted.

As the main body 32 of the inducing member 30 may be a heating wire, or a conductive member with high electrical resistance. Both ends of the main body 32 or the heating wire are connected to a drive circuit 40 including a power source 42 and a switch 44. The drive circuit 40 supplies the inducing member 30 with current flowing through the inducing member 30, in response to the turn-on or the closing operation of the switch 44, and stops supplying current to the inducing member 30 in response to the turn-off or the opening operation of the switch 44. The inducing member 30 generates heat in accordance with the supply of current.

The shape-memory member 20 may be shaped like a wire. The inducing member 30 is arranged close to the shape-memory member 20. The inducing member 30 may be shaped like a coil and the shape-memory member 20 may extend inside the coil-shaped inducing member 30. With this placement, heat generated from the inducing member 30 is transmitted to the shape-memory member 20 with efficiency.

### ANOTHER CONSTITUTION EXAMPLE

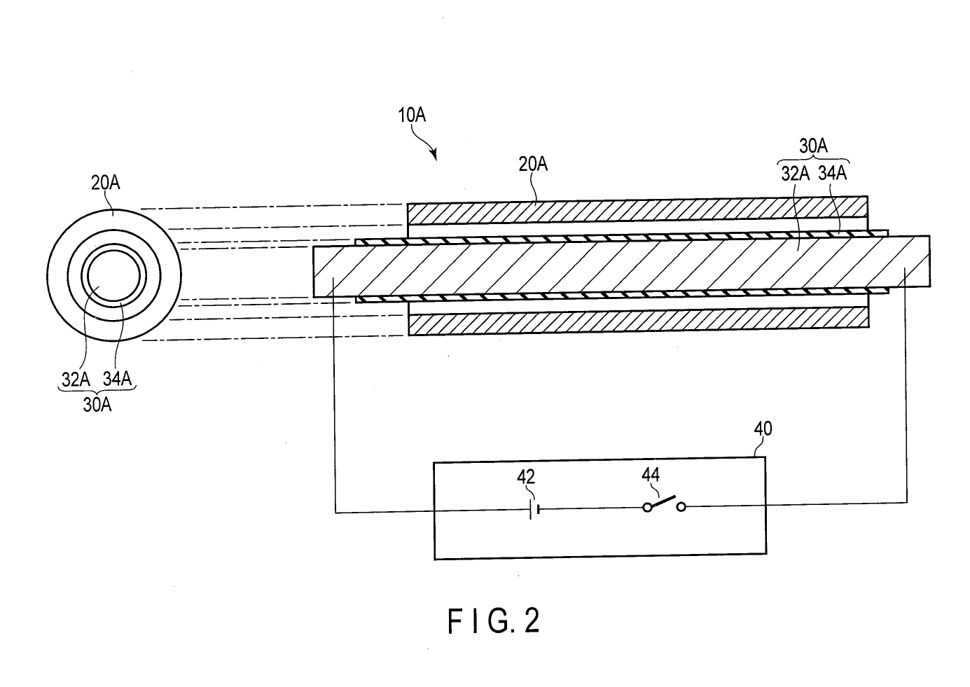

FIG. 2 shows a variable-stiffness actuator according to another embodiment. As shown in FIG. 2, like the variable-stiffness actuator 10, a variable-stiffness actuator 10A includes a shape-memory member 20A that can transit in phase between a first phase and a second phase and an inducing member 30A that causes phase transition between the first phase and the second phase into the shape-memory member 20A.

The shape-memory member 20A has various characteristics similar to those of the shape-memory member 20. Furthermore, the inducing member 30A has various characteristics similar to those of the inducing member 30.

The shape-memory member 20A is shaped like a pipe. The inducing member 30A is shaped like a wire that is easily deformable, and extends inside the shape-memory member 20A. With this placement, heat generated from the inducing member 30 is transmitted to the shape-memory member 20A with efficiency. Since the elastic modulus of the shape-memory member 20A depends upon its radial dimension, the pipe-shaped shape-memory member 20A exhibits an elastic modulus that is higher than that of a solid structure under the same volume condition and thus provides high stiffness.

[Description of Operation of Variable-Stiffness Actuator Alone]

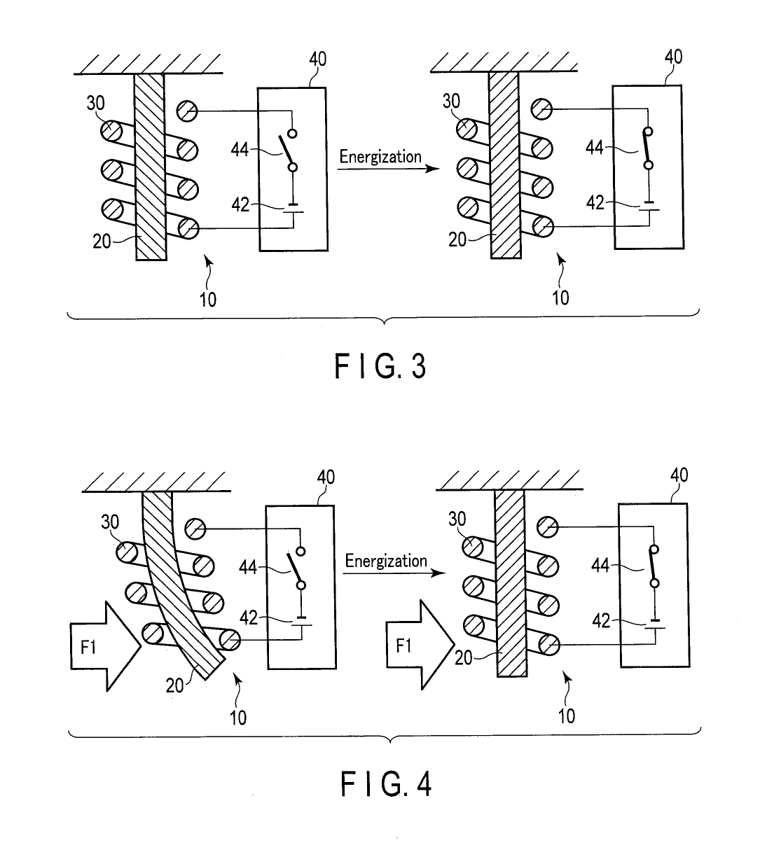

Hereinafter, an operation of the foregoing variable-stiffness actuator will be described with reference to FIGS. 3-8. For convenience of description, it is assumed that an end of the shape-memory member 20 is fixed. It is also assumed that the memorized shape of the shape-memory member 20 is a linear shape. In FIGS. 3-8, the shape-memory member 20 in the flexible state is indicated by upper left hatching and the shape-memory member 20 in the rigid state is indicated by upper right hatching. In FIGS. 3-8, the variable-stiffness actuator 10 shown in FIG. 1 is representatively depicted, and the operation of the variable-stiffness actuator 10A shown in FIG. 2 is similar to that of the variable-stiffness actuator 10.

FIG. 3 shows how the stiffness state of the shape-memory member 20 is varied by switching the switch 44 of the drive circuit 40.

On the left side of FIG. 3, the switch 44 of the drive circuit 40 is in an off state or opened, and the shape-memory member 20 is in the first phase that is the flexible state with a low elastic modulus.

When the switch 44 of the drive circuit 40 is switched to an on state or closed as shown in the right side of FIG. 3, current flows through the inducing member 30, the inducing member 30 generating heat. Accordingly, the shape-memory member 20 transits to the second phase that is the rigid state with a high elastic modulus.

FIG. 4 shows how the stiffness state of the shape-memory member 20 is varied by switching the switch 44 of the drive circuit 40 in a situation where an external force F1 is exerted on the vicinity of the free end of the shape-memory member 20 in a direction perpendicular to the central axis of the shape-memory member 20. The external force F1 is smaller than a restoring force when the shape-memory member 20 will return to its memorized shape.

On the left side of FIG. 4, the switch 44 of the drive circuit 40 is in the off state, and the shape-memory member 20 is in the first phase that is the flexible state. In the first phase, the shape-memory member 20 is in a state in which it is easily deformed by the external force F1. The shape-memory member 20 is bent by the external force F1.

When the switch 44 of the drive circuit 40 is switched to the on state as shown in the right side of FIG. 4, the inducing member 30 generates heat and the shape-memory member 20 transits to the second phase that is the rigid state. In the second phase, the shape-memory member 20 tends to take its memorized shape. In other words, if the shape of the shape-memory member 20 differs from the memorized shape, the shape-memory member 20 will return to the memorized shape. Since the external force F1 is smaller than the restoring force of the shape-memory member 20, the shape-memory member 20 returns to the memorized shape or linear shape against the external force F1.

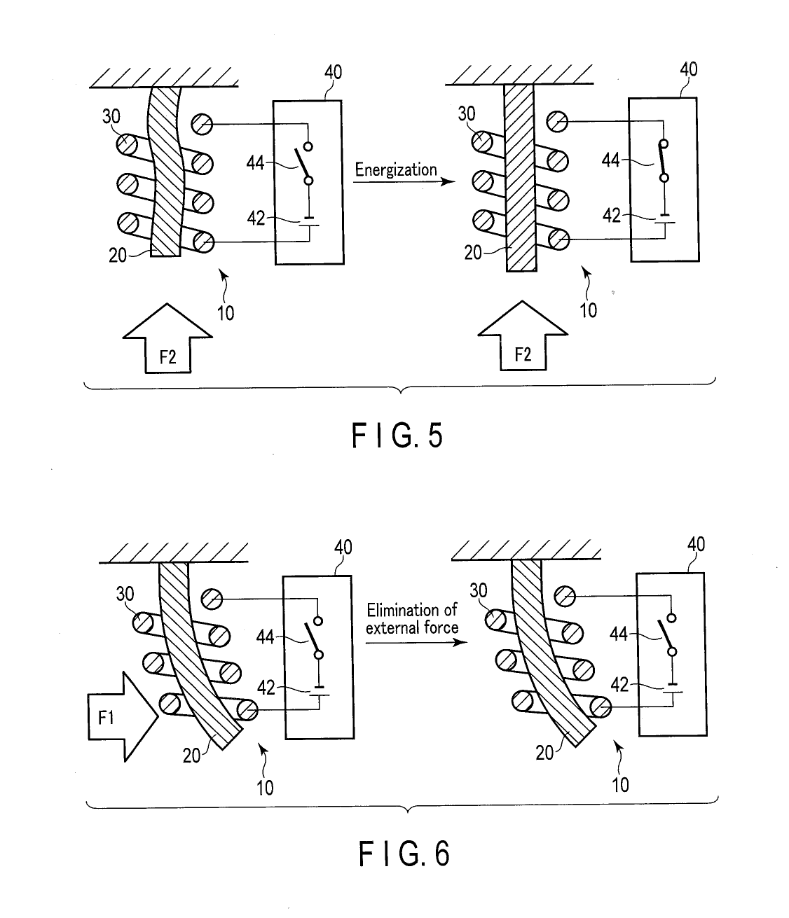

FIG. 5 shows how the stiffness state of the shape-memory member 20 is varied by switching the switch 44 of the drive circuit 40 in a situation where an external force F2 is exerted on the free end of the shape-memory member 20 in a direction parallel to the central axis of the shape-memory member 20. The external force F2 is smaller than the restoring force when the shape-memory member 20 will return to its memorized shape.

On the left side of FIG. 5, the switch 44 of the drive circuit 40 is in the off state, and the shape-memory member 20 is in the first phase that is the flexible state. In the first phase, the shape-memory member 20 is in a state in which it is easily deformed by the external force F2. The shape-memory member 20 is compressed by the external force F2. In other words, the shape-memory member 20 is reduced in its length or its dimension along the central axis with bet.

When the switch 44 of the drive circuit 40 is switched to the on state as shown in the right side of FIG. 5, the inducing member 30 generates heat and the shape-memory member 20 transits to the second phase that is the rigid state. In the second phase, the shape-memory member 20 tends to take its memorized shape. Since the external force F2 is smaller than the restoring force of the shape-memory member 20, the shape-memory member 20 returns to the memorized shape or linear shape against the external force F2.

FIG. 6 shows how the presence and absence of an external force are switched in a situation where the switch 44 of the drive circuit 40 is in the off state and the shape-memory member 20 is in the flexible state. In the first phase, the shape-memory member 20 is in a state in which it is easily deformed by the external force.

On the left side of FIG. 6, the external force F1 is exerted on the vicinity of the free end of the shape-memory member 20 in a direction perpendicular to the central axis of the shape-memory member 20. The shape-memory member 20 is bent by the external force F1.

On the right side of FIG. 6, the external force F1 that has been so far exerted on the shape-memory member 20 is eliminated. The shape-memory member 20 remains bent after the external force F1 is eliminated.

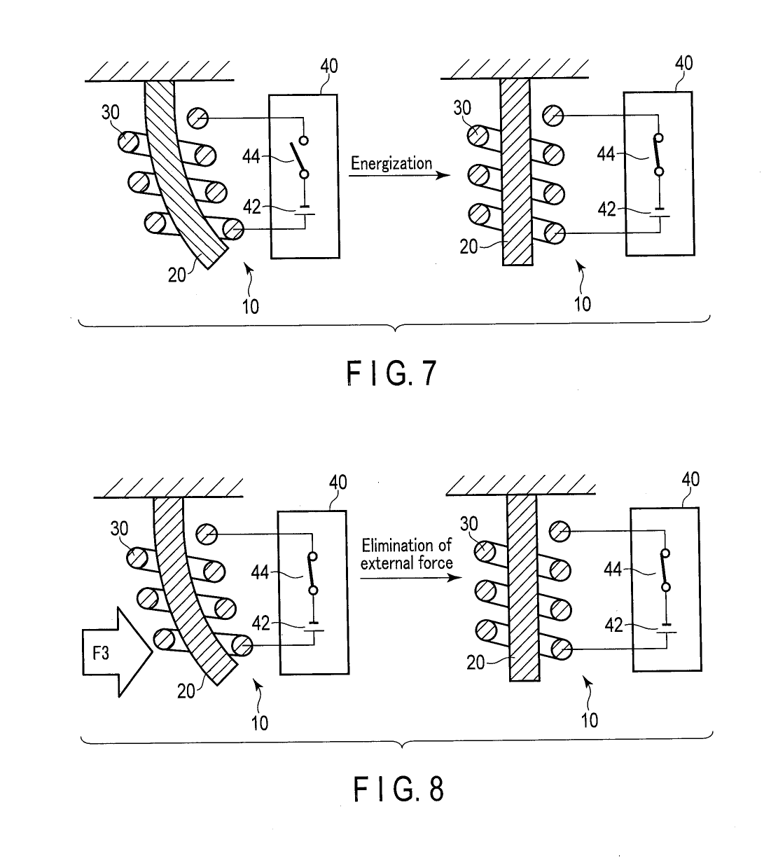

FIG. 7 shows how the stiffness state of the bent shape-memory member 20 is varied from the flexible state to the rigid state by switching the switch 44 of the drive circuit 40.

The left side of FIG. 7 shows the same state as that of the right side of FIG. 6 and, in other words, the shape-memory member 20 is bent by the external force F1, and then remains bent after the external force F1 is eliminated.

When the switch 44 of the drive circuit 40 is switched to the on state as shown in the right side of FIG. 7, the inducing member 30 generates heat and the shape-memory member 20 transits to the second phase that is the rigid state. In the second phase, since the shape-memory member 20 tends to take its memorized shape, the shape-memory member 20 returns to the memorized shape or linear shape.

FIG. 8 shows how the presence and absence of an external force are switched in a situation where the switch 44 of the drive circuit 40 is in the on state and the shape-memory member 20 is in the second phase that is the rigid state. In the second phase, the shape-memory member 20 tends to take its memorized shape.

The left side of FIG. 8 shows how an external force F3 is exerted on the vicinity of the free end of the shape-memory member 20 in a direction perpendicular to the central axis of the shape-memory member 20. The external force F3 is greater than a restoring force when the shape-memory member 20 will return to its memorized shape. Though the shape-memory member 20 will return to its memorized shape against the external force F3, since the external force F3 is greater than the restoring force of the shape-memory member 20, the shape-memory member 20 is bent by the external force F3.

On the right side of FIG. 8, the external force F3 that has been so far exerted on the shape-memory member 20 is eliminated. Since the external force F3 that is greater than the restoring force of the shape-memory member 20 is eliminated, the shape-memory member 20 has returned to its memorized shape or linear shape.

[Description of Operation and Attachment Method of Variable-Stiffness Actuator]

The foregoing variable-stiffness actuator 10 (10A) is installed in a flexible member without restricting both ends of the shape-memory member 20 (20A). For example, the variable-stiffness actuator 10 (10A) is placed in a limited space of the flexible member with a small clearance so that an end or both ends of the shape-memory member 20 (20A) are a free end or free ends.

Herein, the limited space means space capable of exactly containing the variable-stiffness actuator 10 (10A). Thus, even though one of the variable-stiffness actuator 10 (10A) and the flexible member is slightly deformed, it can contact the other and give an external force.

For example, the flexible member may be a tube having an inner diameter that is slightly larger than the outer diameter of the variable-stiffness actuator 10 (10A), and the variable-stiffness actuator 10 (10A) may be placed inside the tube. Without limiting to this, the flexible member has only to have space that is slightly larger than the variable-stiffness actuator 10 (10A).

When the shape-memory member 20 (20A) is in the first phase, the variable-stiffness actuator 10 (10A) provides lower stiffness for the flexible member and is easily deformed by an external force exerted on the flexible member, or force capable of deforming the shape-memory member 20 (20A).

When the shape-memory member 20 (20A) is in the second phase, the variable-stiffness actuator 10 (10A) provides higher stiffness for the flexible member and tends to return to its memorized shape against an external force exerted on the flexible member, or force capable of deforming the shape-memory member 20 (20A).

For example, the phase of the shape-memory member 20 (20A) is switched between the first and second phases by the drive circuit 40 switches, so that the stiffness of the flexible member is switched.

In addition to the switching of stiffness, in a situation where an external force is exerted on the flexible member, the variable-stiffness actuator 10 (10A) also serves as a bidirectional actuator that switches the shape of the flexible member. In another situation where no external force is exerted on the flexible member but the flexible member is deformed in the first phase before the phase of the shape-memory member 20 (20A) is switched to the second phase, it also serves as a unidirectional actuator that returns the shape of the flexible member to the original.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A variable-stiffness actuator capable of providing different stiffnesses for a flexible member, comprising:
a shape-memory member that can transit in phase between a first phase and a second phase, the shape-memory member taking a flexible state in which the shape-memory member is easily deformable by an external force when it is in the first phase, so as to provide lower stiffness for the flexible member, the shape-memory member taking a rigid state in which the shape-memory member tends to take a memorized shape memorized beforehand against an external force when it is in the second phase, so as to provide higher stiffness for the flexible member; and
an inducing member that causes phase transition between the first phase and the second phase into the shape-memory member, the shape-memory member being arranged in the flexible member with at least one free end.

2. The variable-stiffness actuator according to claim 1, wherein the inducing member has performance of generating heat, and the shape-memory member has properties of transiting in phase from the first phase to the second phase in response to heating of the inducing member.

3. The variable-stiffness actuator according to claim 1, wherein the shape-memory member and the inducing member are each constituted chiefly from a conductive material, and the variable-stiffness actuator further comprises an insulation member that prevents a short circuit from occurring between the shape-memory member and the inducing member.

4. The variable-stiffness actuator according to claim 1, wherein the shape-memory member is shaped like a wire and the inducing member is arranged close to the shape-memory member.

5. The variable-stiffness actuator according to claim 4, wherein the inducing member is shaped like a coil and the shape-memory member extends inside the inducing member.

6. The variable-stiffness actuator according to claim 1, wherein the shape-memory member is shaped like a pipe.

7. The variable-stiffness actuator according to claim 6, wherein the inducing member extends inside the shape-memory member.

8. The variable-stiffness actuator according to claim 1, wherein the shape-memory member is constituted chiefly from an alloy including NiTi.

9. The variable-stiffness actuator according to claim 1, wherein the inducing member has properties of generating heat upon receipt of current flowing therethrough.

10. The variable-stiffness actuator according to claim 1, wherein the memorized shape is a linear shape.
